(19) United States
(12) Patent Application Publication
(10) Pub. No.: US 2023/0301737 A1
(43) Pub. Date: Sep. 28, 2023

(54) EMERGENCY RELEASE AND STERILE BARRIERS FOR ROBOTIC SURGERY SYSTEMS

(71) Applicant: Titan Medical Inc., Toronto (CA)

(72) Inventors: Ryan Goffrid Springer, Chapel Hill, NC (US); Matthew Alexander Morales, Chapel Hill, NC (US); Hans Christian Pflaumer, Raleigh, NC (US); Jessica Alzamora, Raleigh, NC (US); Sukie Whitehall, Toft (GB)

(21) Appl. No.: 18/188,737

(22) Filed: Mar. 23, 2023

Related U.S. Application Data
(60) Provisional application No. 63/323,225, filed on Mar. 24, 2022.

(51) Int. Cl. A61B 34/00

(52) U.S. Cl. CPC A61B 34/74; A61B 2034/743

(57) ABSTRACT

An instrument manipulator for a robotic surgery system can include a housing configured to support a surgical instrument. The instrument manipulator can include an actuator configured to move the housing forward and backward. The instrument manipulator can include a release connected to the housing and configured to engage with the actuator in a first configuration to allow the housing and the at least one surgical instrument to move forward responsive to activation of the actuator. The release can be configured to disengage from the actuator in a second configuration to allow a user to manually retract the housing and the at least one surgical instrument. The instrument manipulator can include a user interface positioned at least partially on an exterior surface of the housing and configured to permit the user to transition the release from the first configuration to the second configuration.

### TECHNICAL FIELD

This disclosure relates generally to robotic surgery systems and more particularly to an emergency release of surgical instruments of a robotic surgery system.

### DESCRIPTION OF RELATED ART

A robotic surgery system can employ one or more articulating instruments to perform surgical functions within a surgical site in a body cavity of a patient. The articulating instruments may be automatically controlled by the robotic surgery system in response to inputs from an input device having some means of sensing movements of a surgeon's hands. For example, the input device may include a pair of hand controllers that are grasped in the surgeon's hand and moved to cause corresponding movement of the articulating instruments. It is generally desirable that the movement of the articulating instruments closely mimic the surgeon's hand movements so that performing operations. In case of an emergency, it may be necessary to remove the articulating instruments from the surgical site. However, if the robotic surgery system fails, it may not be possible to remove the articulating instruments using an automated approach provided by the system. The present disclosure overcomes these and other problems associated with system and patient safety.

### SUMMARY

An instrument manipulator for a robotic surgery system can include a housing configured to support at least one surgical instrument. The instrument manipulator can include an actuator configured to move the housing forward toward a surgical site and backward away from the surgical site. The instrument manipulator can include a release connected to the housing. The release can be configured to engage with the actuator in a first configuration to allow the housing and the at least one surgical instrument to move forward toward the surgical site and backward away from the surgical site responsive to activation of the actuator. The release can be configured to disengage from the actuator in a second configuration to allow a user to manually move the housing and the at least one surgical instrument backward away from the surgical site. The instrument manipulator can include a user interface positioned at least partially on an exterior surface of the housing and configured to permit the user to transition the release from the first configuration to the second configuration, thereby facilitating retraction of the at least one surgical instrument away from the surgical site.

The instrument manipulator of any of the preceding paragraphs and/or any of the instrument manipulators disclosed herein can include one or more of the following features. Manual movement of the housing backward may not be permitted in the first configuration as a result of the engagement of the release with the actuator. The actuator includes a leadscrew. The instrument manipulator can include a motor configured to rotate the leadscrew to cause the leadscrew to advance and retract the housing. The release can include at least one of a half nut, a dual split nut, a torsion spring, or a radial split nut. The release can include a pin and at least one spring. The at least one spring can be configured to extend the pin vertically upward when the release is in the second configuration. The instrument manipulator can include a guide configured to engage with the pin to facilitate retraction of the housing away from the surgical site. The guide can include a cutout configured to limit the movement of the pin. The guide can include a rail. The user interface can include a button.

Disclosed is a central unit that includes at least one instrument manipulator of any of the preceding paragraphs and/or any of the instrument manipulators disclosed herein.

Disclosed are methods of operating the instrument manipulator of any of the preceding paragraphs and/or any of the instrument manipulators disclosed herein.

A method of manually retracting an instrument manipulator of a robotic surgery system can include activating an emergency release of the instrument manipulator to cause the emergency release to disengage from an actuator of the instrument manipulator. The method can include, while the emergency release is activated, manually moving the instrument manipulator and at least one surgical instrument supported by the instrument manipulator backward away from a surgical site.

The method of any of the preceding paragraphs and/or any of the methods disclosed herein can include one or more of the following features. Activating the emergency release of the instrument manipulator can include pressing a button located on an exterior surface of the instrument manipulator. The method can include reengaging the emergency release to the actuator by releasing the button, thereby allowing manipulation of the at least one surgical instrument by the robotic surgery system. The actuator can include a leadscrew. The emergency release can include a ping configured to be biased into a deactivated configuration by a spring. In the deactivated configuration, manual movement of the instrument manipulator is prevented. The emergency release can include at least one of a half nut, a dual split nut, a torsion spring, or a radial split nut. The method can include removing the at least one surgical instrument from the instrument manipulator subsequent to manually moving the instrument manipulator. The method can include removing the at least one surgical instrument from the instrument manipulator subsequent to manually retracting the instrument manipulator to a threshold distance away from the surgical site. The threshold distance can be defined by a stopper of the instrument manipulator configured to limit backward movement of the at least one surgical instrument.

In some cases, a robotic surgery system as described and/or illustrated is provided. In some implementations, one or more emergency release mechanisms as described and/or illustrated are provided. In some cases, one or more sterile barriers as described and/or illustrated are provided. In some cases, one or more sterile adapters as described and/or illustrated are provided. In some cases, one or more kits as described and/or illustrated are provided.

In some cases, a method of using and/or operating a robotic surgery system or any of its components as described and/or illustrated is provided. In some instances, methods for operating one or more emergency release mechanisms as described and/or illustrated are provided. In some cases, methods of preparing a robotic surgery apparatus for a medical procedure as described and/or illustrated are provided. Any of such methods can include positioning one or more sterile barriers and/or adapters on any of the components of the robotic surgery system (or covering any of the components with any of the sterile barriers) as described and/or illustrated.

Any of the methods of any of the preceding paragraphs and/or described herein can be used with any of the emergency release mechanisms and/or robotic surgery systems. Any of the methods of any of the preceding paragraphs and/or described herein can be used with any of sterile barriers, adapters, kits, and/or robotic surgery systems.

### BRIEF DESCRIPTION OF THE DRAWINGS

Embodiments of the present disclosure will now be described hereinafter, by way of example only, with reference to the accompanying drawings in which:

FIGS. 1A-1C illustrate a robotic surgery system;

FIG. 2A-2C illustrate a central unit and instruments of a robotic surgery system;

FIGS. 3A-3L and 4A-4D illustrate emergency release mechanisms for a robotic surgery system and methods of removing one or more surgical instruments;

FIG. 5A illustrates a central unit of a robotic surgery system;

FIG. 5B illustrates a sterile barrier positioned on a central unit of a robotic surgery system; and

FIG. 5C illustrates the sterile barrier.

### DETAILED DESCRIPTION

### Overview of Robotic Surgery System

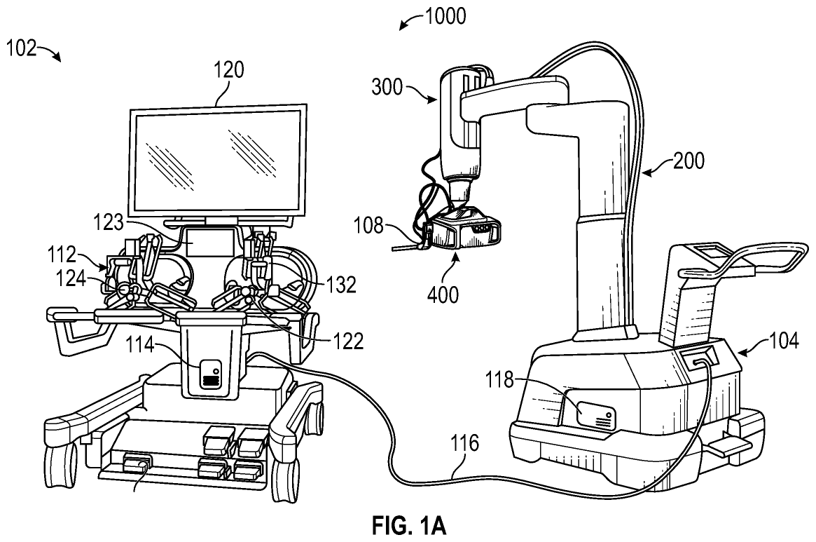

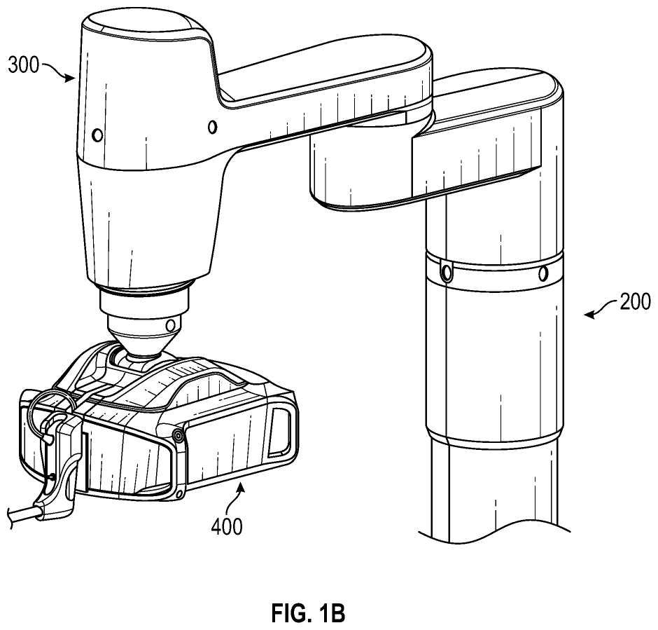

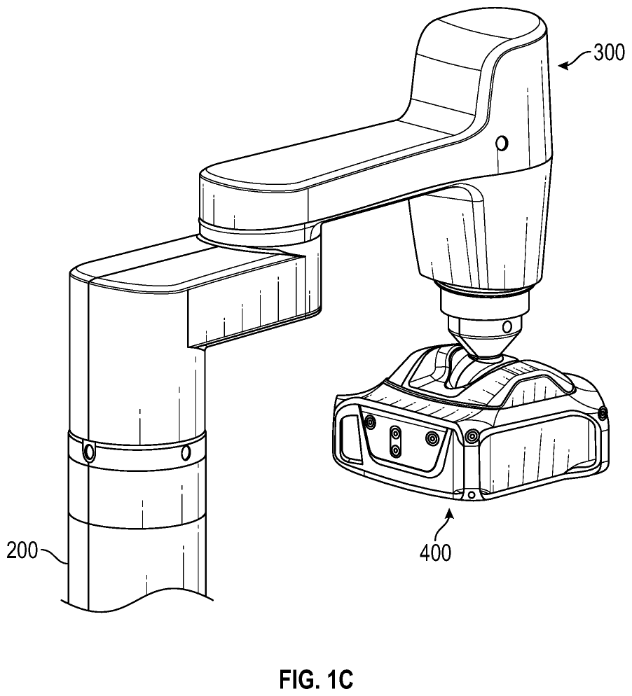

Referring to FIG. 1A, a robotic surgery system is shown generally at 1000. In some implementations, the robotic surgery system 1000 can be configured to facilitate a medical procedure (for example, surgery) performed via one or more incisions. For a single incision, a single access port can be inserted into the incision to provide access for one or more instruments (sometimes referred to as surgical instruments, tools or surgical tools) or cameras (or other non-tissue manipulating equipment, including advanced visualization equipment).

The system 1000 can include a workstation 102 and a patient cart 104. One or more of the patient cart 104 or the workstation 102 can be moveable. The patient cart 104 can include a manipulator unit or central unit 400 to which one or more instrument insertion and visualization devices 108 can be attached or mounted. The central unit 400 can be supported by an elevating linkage assembly 300 (sometimes referred to as an arm) connected to a boom arm assembly 200 (sometimes referred to as a column) of the patient cart. The central unit 400 can be moveable, such as in three dimensions, to facilitate desired positioning of one or more surgical instruments or cameras. Movement of the central unit 400 can be facilitated by one or more of the arm 300 and column 200. For example, the column 200 can facilitate vertical positioning of the central unit 400, and the arm 300 can facilitate further vertical positioning in addition to lateral movement and rotation of the central unit 400.

The instrument insertion and visualization device 108, which can be removably mounted to the central unit 400, can support at least one surgical instrument and one or more cameras (not shown) that image a site of interest, such as a surgical site. The instrument insertion and visualization device 108 can support two or more instruments (not shown). The one or more cameras can include a primary camera and at least one secondary camera. The primary camera and the secondary camera may provide different viewing angles, perform different functions and/or produce different images. At least one of the primary camera and the secondary camera may be a two-dimensional (2D) or a three-dimensional (3D) camera.

The workstation 102 can include an input device 112 that receives operator input and produces input signals and may also be configured to generate feedback to the operator or user. The feedback can be visual, auditory, haptic, or the like. The input device 112 can be implemented using a haptic interface available from Force Dimension, of Switzerland, for example. The operator can be a surgeon.

The workstation 102 can further include electronic circuitry 114 in communication with the input device for receiving the input signals and generating control signals for controlling the robotic surgery system, which can be transmitted to the patient cart 104 via an interface cable 116. In some cases, data transmission can be wireless and the interface cable 116 may not be present. The electronic circuitry 114 can include one or more processors or controllers. The electronic circuitry 114 can function as a master for controlling movement of one or more surgical instruments or cameras mounted to the patient cart 104. The patient cart can include electronic circuitry 118, which can include one or more processors or controllers. The electronic circuitry 118 can function as a slave and be controlled by the electronic circuitry 114. Communication between the electronic circuitry 114 of the workstation 102 and the electronic circuitry 118 of the patient cart 104 may wired (such as, via the cable 116) or wireless. The workstation 102 may be located remotely from the patient cart 104, such as outside the operating room or in a non-sterile area of the operating room.

The input device can include a right input device 132 and a left input device 112 for controlling respective right and left instruments (not shown). The right input device 132 can include a right hand controller 122 (sometimes referred to as a hand grip or handpiece), and the left input device 112 can include a left hand controller 124. The right and left hand controllers 122 and 124 can be mechanically and/or electrically coupled to the respective input devices 132 and 112. Alternatively, the right and left hand controllers 122 and 124 may be wirelessly coupled to the respective input devices 132 and 112 or may be wireless coupled directly to the workstation 102. The right and left hand controllers 122 and 124 can be grasped by the operator's hands and moved to produce input signals at the respective input devices 132 and 112.

In some cases, when there are two instruments at the patient cart 104, the right and left hand controllers 122 and 124 may respectively control the two instruments. In some cases, when there are more than two instruments, the right and left hand controllers 122 and 124 may be used to select two of the multiple instruments that the operator wishes to use at any given time. In some cases, when there is only one instrument, one of the right and left hand controllers 122 and 124 may be used to select the single instrument.

The input devices 132 and 112 may generate input signals representing positions of the hand controllers 122 and 124 within an input device workspace (not shown). In some cases where the input devices 132 and 112 are coupled directly and wirelessly to the workstation, they would include the necessary sensors to allow wireless control such as an accelerometer, a gyroscope and/or magnetometer. In other cases, a wireless connection of the input devices 132 and 112 to the workstation 102 may be accomplished by the use of camera systems alone or in combination with the described sensors. Such sensors for wireless functionality may also be placed in each handpiece to be used in conjunction with the input devices 132 and 112 to independently verify the input device data. The electronic circuitry 114 can be in communication with the input devices 132 and 112 for receiving the input signals.

The electronic circuitry 118 of the patient cart 104 can receive control signals from the electronic circuitry 114 and produce slave control signals operable to control the instrument insertion and visualization device 108 and one or more instruments (and their respective end effectors) and/or cameras during a surgical procedure. The one or more instruments can include dexterous tools, such as grippers, needle drivers, staplers, dissectors, cutters, hooks, graspers, scissors, coagulators, irrigators, and suction devices used for performing a surgical procedure (such as a laparoscopic surgical procedure). While both master and slave electronic circuitry 114 and 118 are illustrated, in some cases a single electronic circuitry may be used to perform both master and slave functions.

The workstation 102 can also include a user interface, such as a display 120 in communication with the electronic circuitry 114 for displaying information (such as, body cavity images) for a region or site of interest (for example, a surgical site, a body cavity, or the like) and other information to the operator. The display 120 can display real time images or other graphical depictions of a surgical site produced by one or more cameras of the visualization device (not shown) and/or one or more cameras of the instrument insertion and visualization device 108. The workstation 102 may include right and left graphical depictions (not shown) displayed on the display 120 respectively for the right and left side instruments (not shown). The graphical depictions may be displayed at a peripheral region of the display 120 to prevent obscuring a live view of the surgical workspace also displayed on the display. The display 120 may further be operable to provide other visual feedback or instructions to the user.

The workstation 102 can include a second auxiliary display 123 to display auxiliary surgical information to the user, for example, patient medical charts, pre-operation images and surgical data. In some cases, the auxiliary display 123 may be a touch display and may also be configured to display graphics representing additional inputs for controlling the workstation 102, the patient cart 104, and/or specific functions thereof.

The workstation 102 can also include one or more controllers, such as one or more footswitches or pedals 126, for controlling the robotic surgery system. For example, one or more pedals 126 can include a clutch pedal that allows repositioning the hand controllers 122 or 124 without corresponding movement of the respective associated instrument. The clutch pedals 126 can provide input signals to the electronic circuitry 114, and the electronic circuitry may inhibit movement of the associated instrument while the footswitch 126 is depressed.

FIG. 1B illustrates a close-up front perspective view of a portion of the patient cart 104. The illustrated portion includes the central unit 400 (which can be shaped as a hexahedron), the arm 300, and an upper portion of the column 200. FIG. 1C illustrates a close-up rear perspective view of the portion of the patient cart 104 illustrated in FIG. 1B. FIGS. 1A-1C illustrate an example of a robotic surgery system and its components, and certain elements may be removed, other elements added, two or more elements combined, or one element can be separated into multiple elements depending on the specification and requirements of the robotic surgery system.

Additional details of the robotic surgery system 1000 and its components, including one or more insertion devices, visualization devices, or cameras, are described in U.S. Publication Nos. 2020/0113640, which published on Apr. 16, 2020, 2020/0113414, which published on Apr. 16, 2020, 2020/0269310, which published on Nov. 26, 2020, and 2020/0367979, which published on Nov. 26, 2020, and U.S. Pat. Nos. 10,398,287, 10,426,561, and 10,881,477, the entire disclose of each of which is incorporated by reference and should be considered part of this specification.

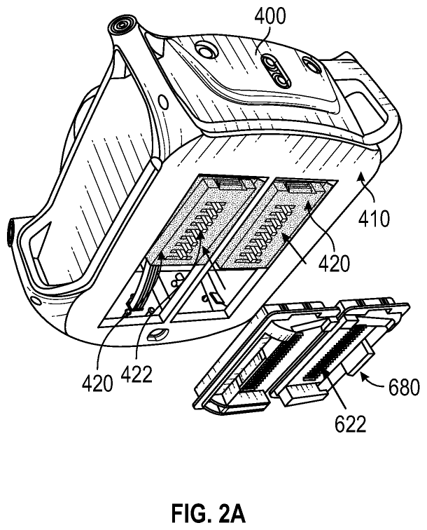

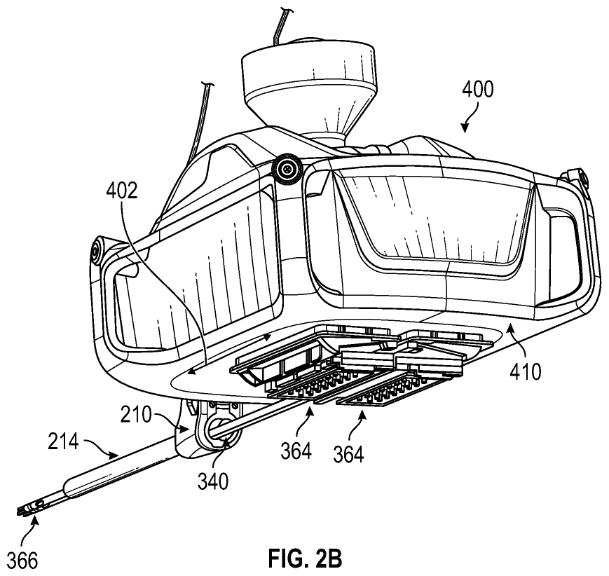

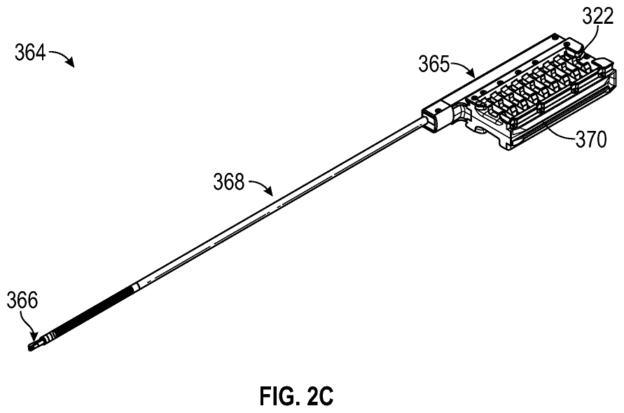

As shown in FIG. 2A, a bottom surface 410 of the central unit 400 can include one or more instrument interfaces 420 configured to support and actuate one or more surgical instruments (as illustrated, for example, in FIG. 2C). Each instrument interface 420 can include a plurality of actuators 422 configured to interface (for example, mate with) with a plurality of actuators of a surgical instrument attached to the instrument interface (for instance, the actuators 322 of the surgical instrument illustrated in FIG. 2C). For example, the protrusion of the actuator 422 can interface with (such as mate with) the opening in an actuator 322 of the surgical instrument (sometimes referred to as surgical instrument actuator). Movement of the actuators 422, such as movement left and right (or up and down, or otherwise from one point to another point), can cause corresponding movement of the actuators of the surgical instrument. This can cause an end effector portion of the instrument (which can be located at the distal end of the instrument) to articulate or move (for example, in three dimensions), change orientation (for example, in three dimensions including rotation), and/or actuate or perform a function (such as, open or close a jaw of a grasper) as needed for performing the medical procedure.

To maintain sterility of the one or more surgical instruments, one or more sterile interfaces 680 (sometimes referred to as covers, interfaces, shields, or adapters) can be attached to or positioned on the instrument interfaces 420 that may not be sterile (for example, due to particular difficulty of sterilizing the plurality of actuators 422). One or more sterile adapters 680 can be removably attached to the instrument interfaces 420, and an instrument can be placed or positioned in contact with the sterile adapter(s) 680 when mounted on the instrument interfaces 420. For example, when a single sterile adapter 680 is used to attach to the instrument interfaces 420, the sterile adapter can be configured to (for example, sized and shaped) to coincide or substantially coincide with the size and shape of the instrument interfaces 420. Sterile adapter 680 can have the same or substantially similar length and width as the instrument interface 420. Additional details of the sterile adapter 680 are disclosed in U.S. Pat. No. 10,881,477, the entire disclose of which is incorporated by reference and should be considered part of this specification.

With reference to FIG. 2B, the central unit 400 is shown in a rear view with surgical instruments 364 attached to the instrument interfaces 420 (or loaded). With reference to FIG. 2C, a surgical instrument 364 can include an instrument housing 365, a shaft 368 connected to the housing, and an end effector 366 connected to the shaft. As illustrated in FIG. 2B, the shaft 368 and the end effector 366 can be loaded through an opening 340 formed in a rear of an insertion device 210 (sometimes referred to as the CIT). The shaft 368 and the end effector 366 can be inserted through a passage, lumen, or channel 214 of the insertion device 210. Left and right surgical instruments 364 can be positioned in such manner. A camera (not shown) can also be positioned in such manner. To insert one or more surgical instruments 364 or the camera into a surgical site (such as, a body cavity), the instrument interfaces 420 can be moved forward (to advance one or more surgical instruments 364) or backward (to retract one or more surgical instruments 364) as shown by the arrow 402.

### Emergency Release

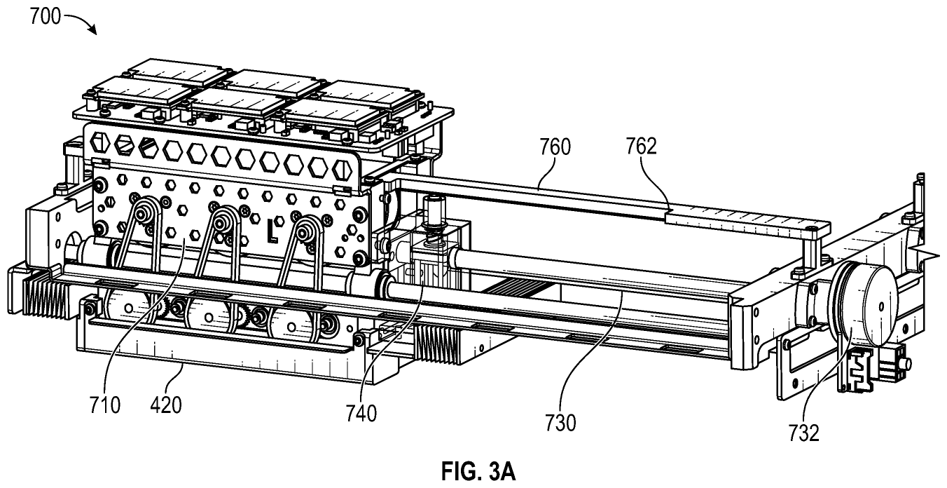

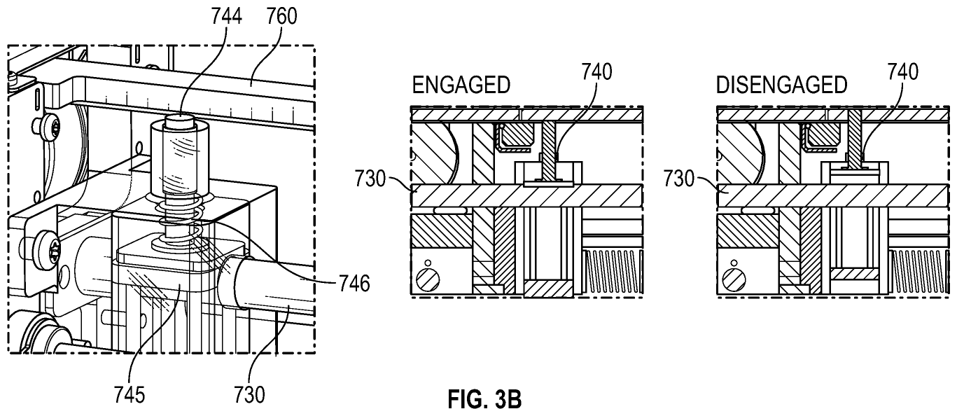

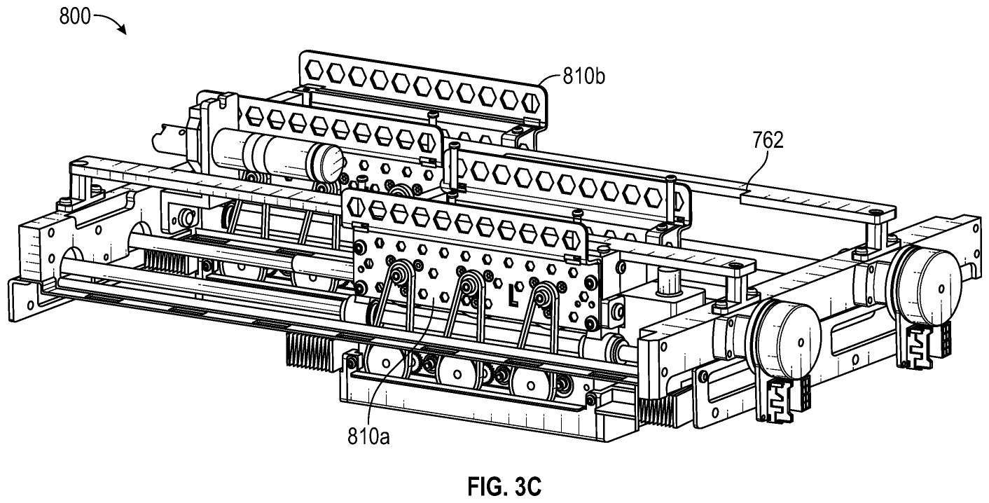

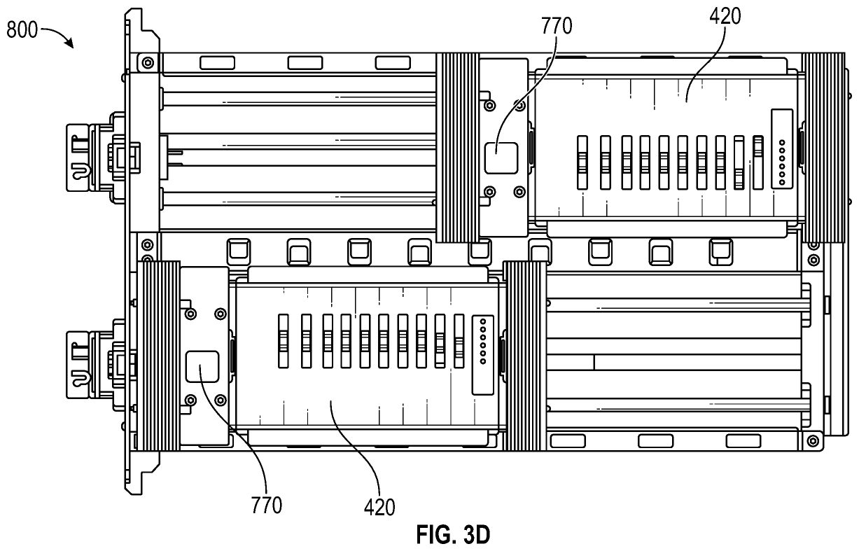

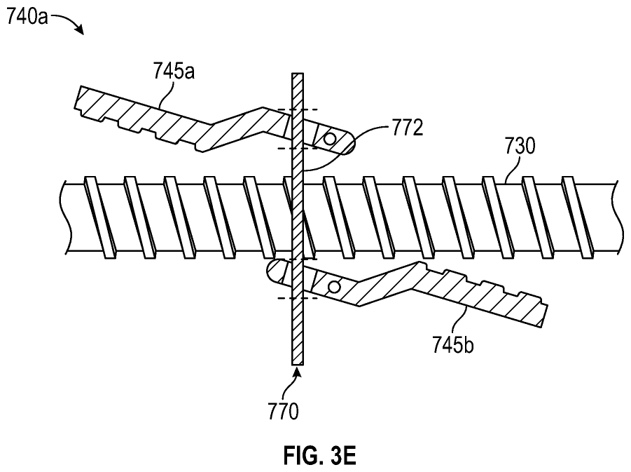

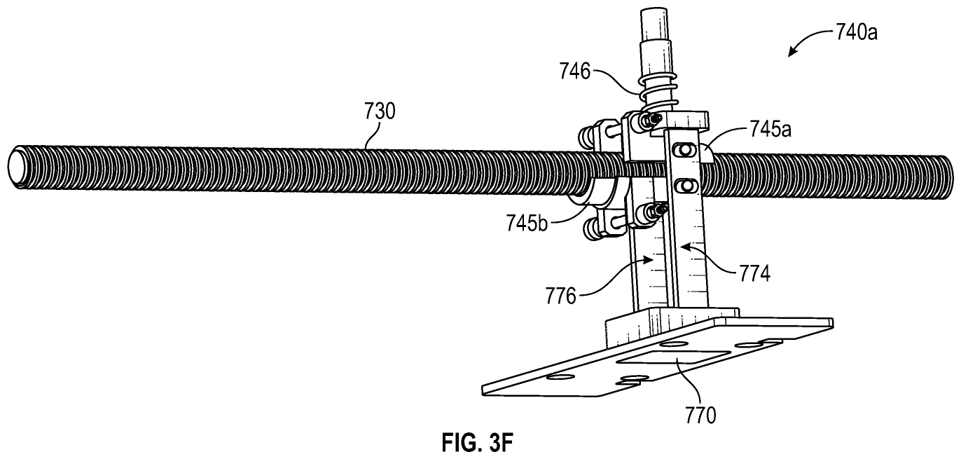

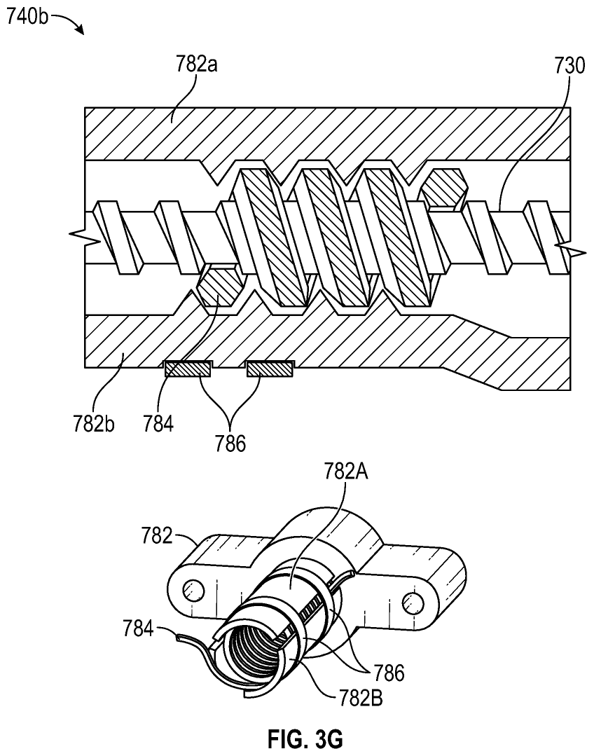

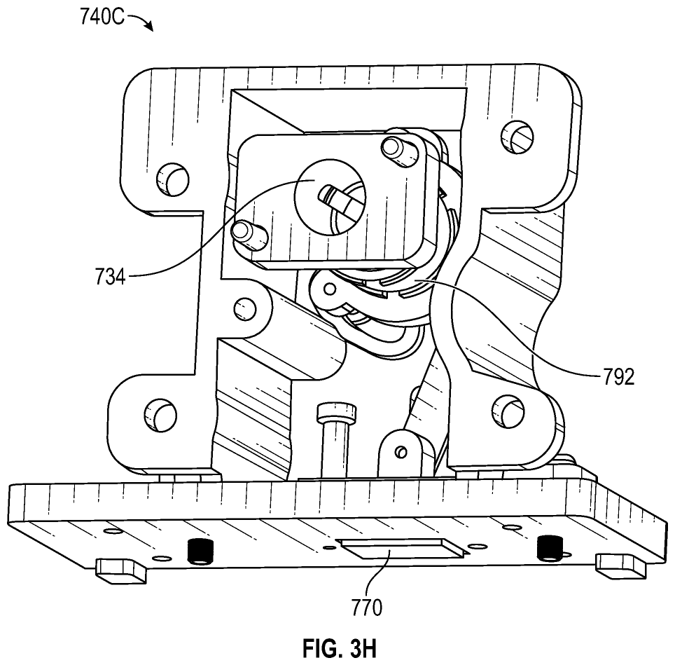

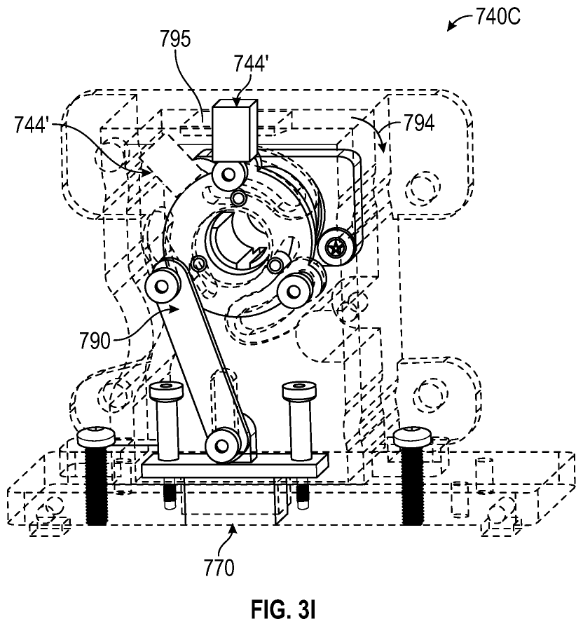

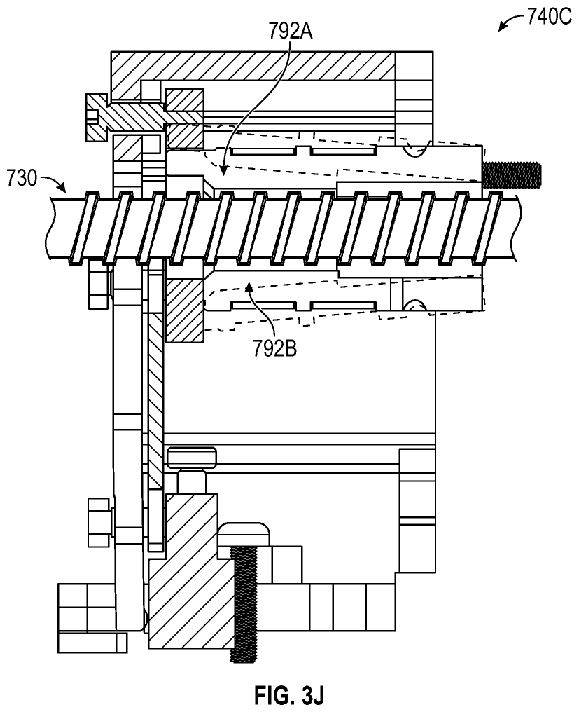

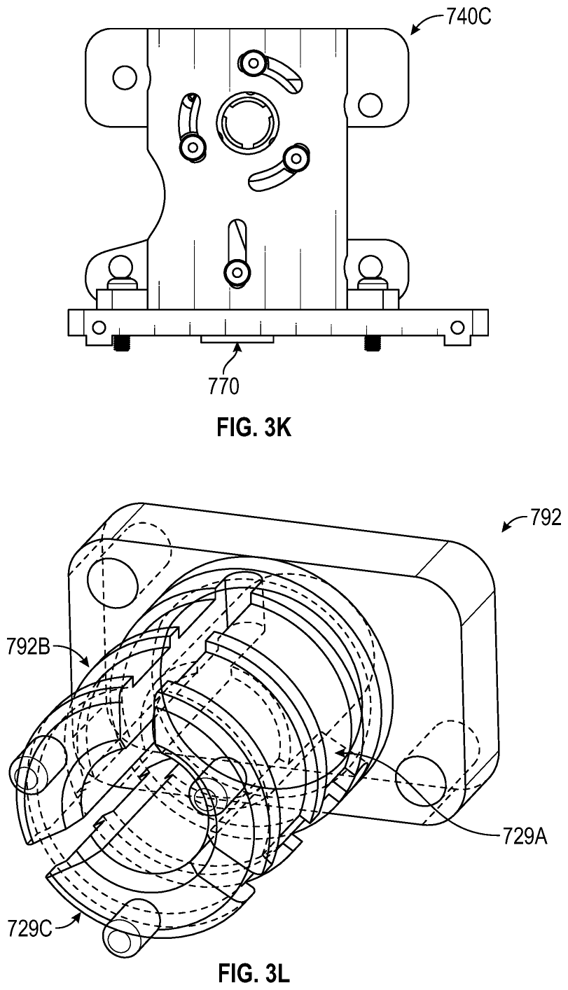

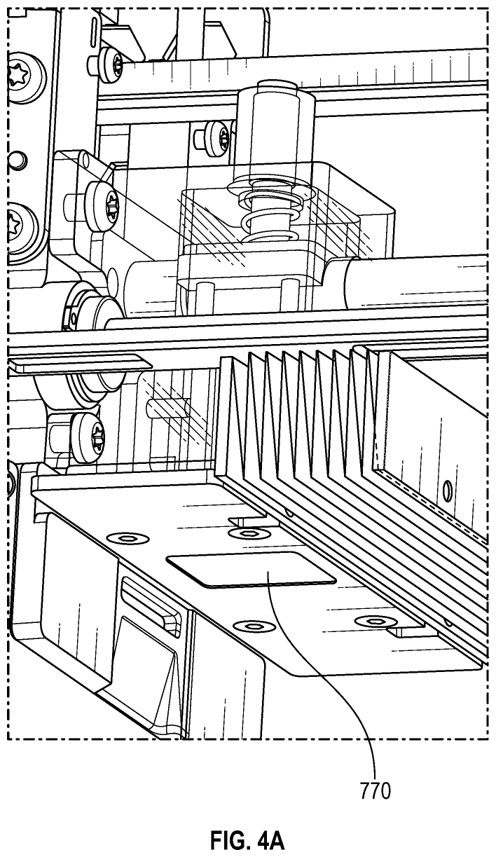

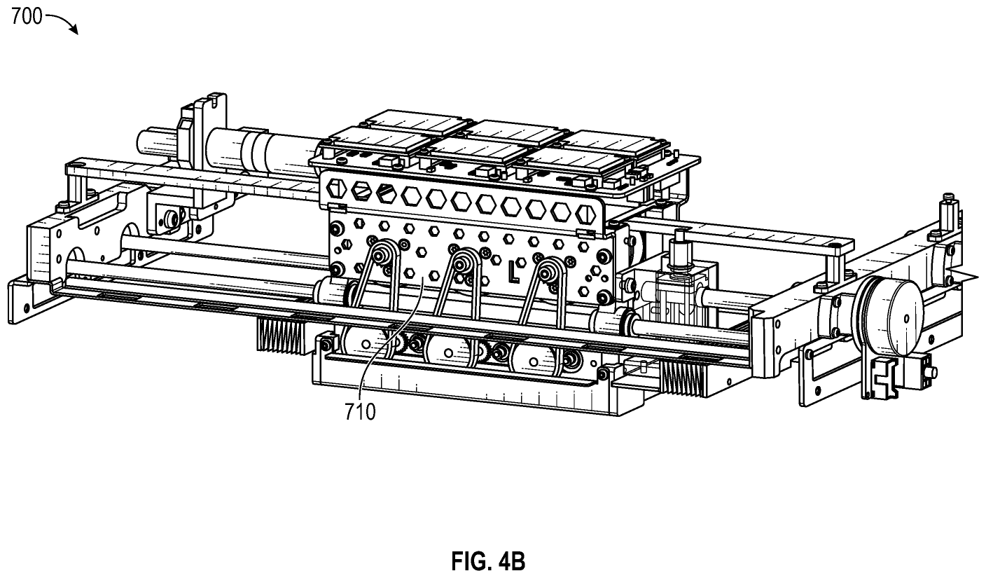

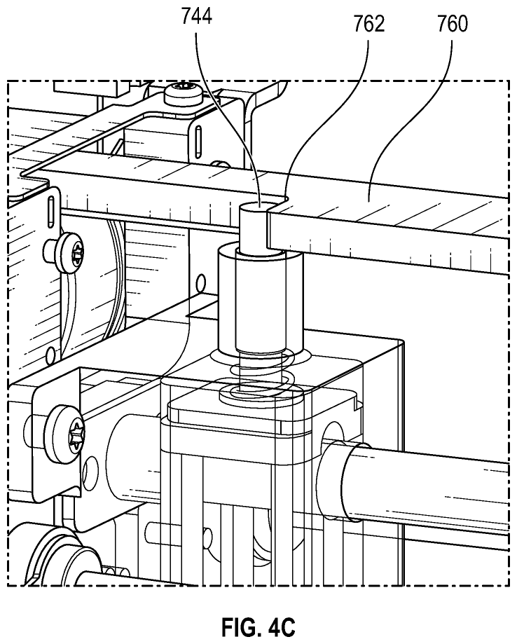

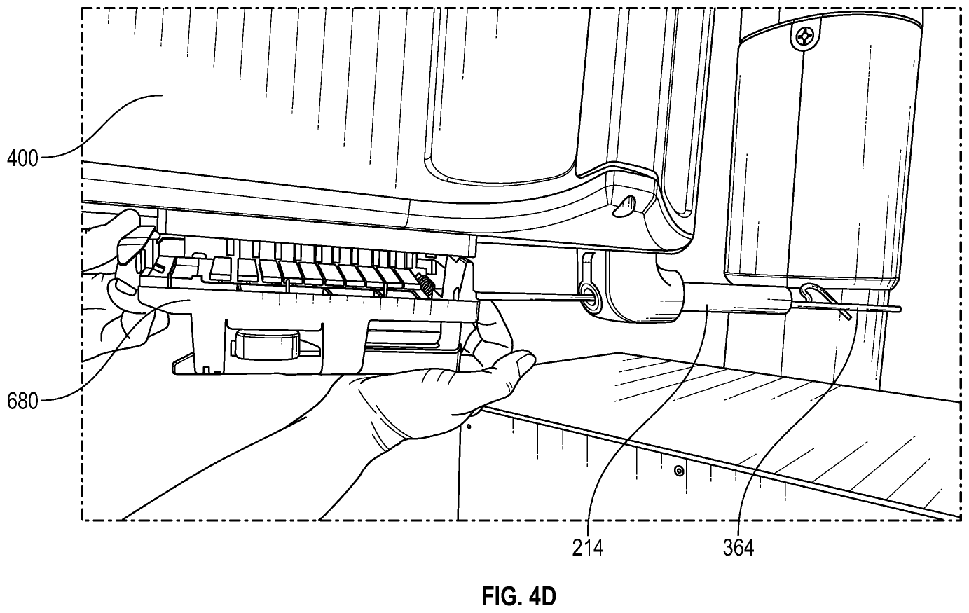

Referring to FIG. 3A, an instrument manipulator (sometimes referred to as an instrument drive unit (IDU)) is shown generally at 700. The instrument manipulator can be part of the central unit 400 and can support and actuate a surgical instrument (for instance, as shown in FIG. 2B in which instrument facings sides of instrument manipulators for left and right instruments are shown as instrument interfaces 420). The instrument manipulator 700 can include a housing 710, a first actuator 730, and an emergency release 740 (sometimes referred to as release or release mechanism 740). In FIG. 3A, the housing 710 of the instrument manipulator 700 is shown in a fully deployed or advanced position (along the direction of the arrow 402), which can place the instruments at or near a surgical site. As described herein, the housing 710 of the instrument manipulator 700 can be configured to support at least one surgical instrument. For example as shown in FIG. 4D, the surgical instrument 364 can be attached, or supported by the instrument interface 420 of the housing 710 via a sterile adapter 680. The first actuator 730 can include a leadscrew and be configured to move the housing 710 forward toward the surgical site and retract the housing 710 backward away from the surgical site. A motor 732 can facilitate motion of the housing 710. For example, the motor 732 can be configured to rotate the first actuator 730 along a longitudinal axis of the first actuator 730 in a first direction, thereby moving the housing 710 forward toward the surgical site or in a second direction (which can be opposite the first direction), thereby moving the housing 710 backward from the surgical site. The release 740 of the instrument manipulator 700 can be connected to the housing 710. In a first configuration (sometimes referred to as default or deactivated configuration), the release 740 can be configured to engage with the first actuator 730. When the release 740 is engaged with the first actuator 730, the housing 710 can move forward and backward responsive to movement of the first actuator 730, as described herein. In the first configuration, manual movement of the housing may be prevented (for example, due to engagement with the first actuator 730). In a second configuration (sometimes referred to as actuated or activated configuration), the release 740 can be configured to disengage from the first actuator 730. When the release 740 is disengaged from the first actuator 730, the housing 710 can be manually moved along a guide 760 (sometimes referred to as a rail 760). The guide 760 can be utilized to encode position of the surgical instrument along the z-axis, which can be necessary for the correct operation of a robotic surgery system. Manual movement of the housing 710, and any surgical instruments supported by the housing 710, from the surgical site can be advantageous in cases of instrument manipulator 700 failure, mechanical failure, loss of power, an emergency, or the like. Advantageously, transitioning the release 740 to the second configuration to allow manual retraction of the surgical instrument(s) from the surgical site can facilitate patient safety in case of a malfunction, failure, or another emergency. The rail 760 can include a stopper 762 configured to limit the movement of the housing 710, for instance, to not damage the housing 710 or any other components of the system. The stopper 762 can prevent manual movement of the housing 710 in one direction (such as, away from the surgical site) while permitting manual movement of the housing 710 in the opposite direction (such as, toward the surgical site). The stopper 762 can include an indentation or cutout along a section of the rail 760 as shown in FIG. 3A. In some implementations, the rail 760 can include a channel or a slot configured to limit the movement of the housing 710.

Referring to FIG. 3B, the release 740 can include at least one of a single split nut (or half nut) mechanism, a dual split nut mechanism, a torsion spring mechanism, and a radial split nut mechanism configured to engage with and/or disengage with the first actuator 730. A second actuator 744 of the release 740 can be connected to a half nut 745 and configured to facilitate engagement and disengagement of the half nut 745 from the first actuator 730 (which, as described herein, can be a leadscrew). The second actuator 744 can be actuated by a button 770 (illustrated in FIGS. 3D and 4A as being positioned on an exterior surface of the instrument manipulator 700) configured to compress a spring 746 of the second actuator 744 and disengage the half nut 745 from the first actuator 730, as shown in FIG. 3B. The second actuator 744 can be configured to maintain the half nut 745 disengaged from the first actuator 730 while the button 770 is pressed, and to engage the half nut 745 with the first actuator 730 when the button 770 is released. The second actuator 744 can be biased (for instance, by the spring 746) to facilitate engagement of the half nut 745 with the first actuator 730 in the default configuration. Pressing the button 770 can cause the second actuator 744 to disengage the half nut 745 from the first actuator 730. In some cases, continuously pressing the button 770 can cause disengagement of the half nut 745 from the first actuator 730. In some implementations, the second actuator 744 can be configured to maintain the half nut 745 disengaged from the first actuator 730 after a single press of the button 770.

The second actuator 744 can include a stopping pin (which can be part of a plunger). The stopping pin can be configured to stay retracted (or in a stand-by position), as shown in FIG. 3B, when the release 740 is in the default configuration (for instance, when the button 770 is not pressed). Activating the release 740, can cause extension of the stopping pin vertically upward (as illustrated in FIG. 4C). For example, when the button 770 is pressed, the plunger and consequently the stopping pin can move upward into a raised position. In such position, the stopping pin can engage the guide 760 to facilitate controlled manual retraction of the instrument manipulator 700. With reference to FIG. 3A, the stopper 762 along the rail 760 can limit manual movement of the housing 710 along the rail 760 in at least one direction by securing the stopping pin in place when the stopping pin comes into contact with the stopper 762 (which can be a cutout in the rail 760). FIGS. 4B and 4C show an example of a stopping pin in the raised position coming into contact with the stopper 762. The motion of the stopping pin can be restricted by the stopper 762 of the rail 760, thereby preventing any further manual movement of the housing 710 along the rail 760 beyond the stopper 762. In FIGS. 4B and 4C, the housing 710 of the instrument manipulator 700 is shown in a fully retracted position away from the surgical site.

The instrument manipulator (or surgical manipulator) can include more than one housing to support multiple surgical instruments. For example, as illustrated in FIG. 3C, an instrument manipulator 800 can include two housings 810a, 810b. Like the housing 710, each housing 810a, 810b, can be configured to support a surgical instrument. Further, like the housing 710, each housing 810a, 810b, can be configured to allow manual movement of each housing 810a, 810b away from a surgical site. The surgical manipulator 800 can include all the components of the surgical manipulator 700 including, but not limited to, the release 740. Thus, the instrument manipulator 800 can operate as the instrument manipulator 700 described herein. As illustrated in FIG. 3D, the instrument manipulator 800 can include at least one button 770 (two buttons are illustrated) configured to activate at least one release 740 (two releases (not shown) are included in the instrument manipulator 800)

FIG. 3E illustrates a release 740A that can be similar to the release 740. The release 740A can include a dual split nut mechanism, which includes a pair of nuts 745A and 745B. In the default configuration of the release 740A, the nut 745A and 745B can be engaged with the first actuator 730 (shown as leadscrew). In the activated configuration (for example, caused by pressing the button 770), the nut 745A and 745B can pivot about a rod 772 and disengage from the first actuator 730. Pressing the button 770 can rotate or linearly move the rod 772 causing the nut 745A and 745B to rotate and disengage from the first actuator.

FIG. 3F illustrates the release 740A in a default configuration in which the nut 745A and 745B is engaged with the first actuator 730. Bars 774 and 776 replace the rod 772 and function similarly to the rod 772.

FIG. 3G illustrates a torsion spring (or torsional spring or helical spring) release 740B that can be similar to the release 740. The release 740B can include a housing (a portion of which can be split into a nut 782 as shown in FIG. 3G) and a torsion spring 784 positioned within the housing of the nut 782. The portion of the housing of the nut 782 enclosing the torsion spring 784 can be split into two (or more) portions 782A and 782B separated by one or more gaps, thus forming a split nut configuration. The portions of the housing of the nut 782 can be held together by one or more banding clamps 786 (two such clamps are illustrated), which can be made from elastic material. The one or more banding clamps 786 may be omitted in implementations in which the housing is made of flexible material and a split nut configuration is utilized. In some cases, a half nut configuration (in which only one of the two portions 782A or 782B is present) may require a retention mechanism of the remaining half and the torsion spring. Torsion spring 784 can function as a nut that engages the first actuator 730 (shown as leadscrew) in the default configuration of the release 740B. When force is applied the ends of the torsion spring 784 as a result of actuating the release 740B (for instance, due to a press of the button 770), the torsion spring 784 can expand radially and disengaging from the first actuator 730. Expansion of the torsion spring 784 can cause the portions 782A and 782B to at least partially come apart (or split open or deflect). The nut 782 can be made at least partially from flexible material such that the portions 782A and 782B can bend. One or more banding clamps 786 can hold the portions 782A and 782B and the torsion spring 784 together. In some implementations, a single body nut 782 can be replaced with two half nuts.

FIGS. 3I to 3L illustrate a radial split nut release 740C, which can be similar to the torsion spring release 740B with the exception of different actuation being utilized. FIG. 3H illustrates a perspective view of the release 740C without the first actuator 730 (which is inserted through the opening 734). FIG. 3K illustrate a rear view of the release 740C. With reference to FIG. 3I, which illustrates a cross-sectional view of the release 740C, a cam mechanism 790 (or cam) can be used to activate the emergency release (the cam mechanism 790 can serve a similar purpose as the second actuator 744). The cam mechanism 790 can be actuated by pressing the button 770 and can rotate (as shown by the arrow 794) to move apart (or open) a split nut mechanism, as illustrated in FIG. 3J. A nut 792 (as shown in FIG. 3H) can include two (or more) portions separated by one or more gaps (thereby forming a split nut configuration). For example, FIG. 3G illustrates two portions 792A and 792B of the nut 792, which in the default configuration engage with the first actuator 730. FIG. 3L illustrates three portions 792A, 792B, and 792C of the nut 792. The portions 792A, 792B, and 792C can include threading that engages with the threading of the first actuator 730, which can be a leadscrew. Responsive to the cam mechanism 790 being activated, the portions 792A, 792B, and 792C can be moved apart (or deflected) to disengage from the first actuator (for instance, as shown in dashed lines for the portions 792A and 792B illustrated in FIG. 3J). The portions 792A, 792B, and 792C can be moved apart by one or more pegs of the cam mechanism 790. The nut 792 can be made at least partially from flexible material such that the portions 792A, 792B, and 792C can bend. One or more banding clamps can be utilized to hold together the portions 792A, 792B, and 792C. In some implementations, a single body nut 792 can be replaced with two half nuts.

In some cases, a stopping pin 744′ can be connected to the cam mechanism 790 and rotated as illustrated. As described herein, the stopping pin 744′ can function as the stopping pin of the second actuator 744 to limit the movement of the housing 710 during manual retraction. The stopping pin 744′ can exit through the opening 795 in a housing of the release 740C.

A method of manually retracting the instrument manipulator 700 can include one or more of the following steps. When the instrument manipulator (such as, 700) is in a fully deployed position, which can be at or near the surgical site, the method can include activating the emergency release 740 to disengage the release from the first actuator 730. In some implementations, prior to activating the emergency release 740, any grip by an end effector of the surgical instrument may need to be released. Activating the emergency release from the first actuator 730 can include pressing the button 770. The button 770 can be positioned on the exterior surface of the instrument manipulator 700 so that it can be accessed by a user. Subsequent to activation of the emergency release 740, the user can manually move the instrument manipulator 700 (for instance, along the rail 760) and away from the surgical site. As described herein, the user may need to continue holding down the button to maintain the release 740 in activated state. The method can include releasing the button 770 when the instrument manipulator 700 is in a fully retracted position (which can be away from the surgical site). As described herein, such position can be reached responsive to contacting the stopper 762. Releasing the button 770 can disengage the release 740 thereby preventing manual movement of the instrument manipulator 700.

The method can also include removing one or more surgical instruments 364 from the instrument manipulator 700. To protect the patient (for instance, from foreign particles falling into the surgical site), the method can include removing the one or more surgical instruments 364 when the instrument manipulator 700 is in the fully retracted position. In some cases, the one or more surgical instruments 364 can be removed from the instrument manipulator 700 even when the instrument manipulator 700 is not in the fully retracted position. Removing the one or more instruments from the instrument manipulator can include releasing one or more sterile adapters 680 from a housing of the instrument manipulator 700.

It may be advantageous to facilitate removal of one or more surgical instruments 364 from the surgical site of in case of malfunction of the robotic surgery system (or any of its components), loss of power, emergency, or the like. With reference to FIG. 2A (which illustrates the central unit 400 without a sterile barrier), the sterile adapter 680 can include a plurality of actuator covers 622 corresponding in size and shape to the plurality of actuators 422. During the medical procedure, the actuators 422 (and actuator covers), which can be configured to move independently of one another, may not be in an aligned position due to the manipulation of the end effector portion of the surgical instrument. Because of the misalignment, it may not be possible to remove the surgical instrument by simply pulling out the instrument from the sterile adapter 680. Instead, the sterile adapter 680 can be configured to allow the surgical instrument 364 to be removed when the robotic surgery system is in use by removing the sterile adapter 680 with the surgical instrument 364 installed. The surgical instrument 364 can become straightened (as shown for one of the instruments in FIG. 4D) when disengaged from the instrument interface 420. Additional implementations of removing the surgical instrument 364 (such as, by rotating the surgical instrument 364 within the sterile adapter 680) are disclosed in U.S. Pat. No. 10,881,477, the entire disclose of which is incorporated by reference and should be considered part of this specification.

FIG. 4D illustrates removal of the surgical instrument 364 when the instrument manipulator 700 in a fully retracted position, The surgical instrument 364 can be removed from the instrument manipulator 700 by detaching the sterile adapter 680 from the instrument manipulator 700. To minimize the risk of injury to the patient or damage to the central unit 400 or the surgical instrument 364, it may be beneficial to remove the sterile adapter 680 from the housing 710 once the housing 710 is in the fully retracted position (or, in some cases, prior to the housing being in the fully retracted position, such as, at a set distance prior to the housing being in the fully retracted position). When the sterile adapter 680 is detached from the housing 710 of the instrument manipulator 700, the surgical instrument 364 can be configured to straighten (as is illustrated with the closer instrument shown in FIG. 4D). This can allow the surgical instrument to be safely removed from an insertion device 210 (which can include one or more lumens or channels for guiding the surgical instruments).

### Additional Approaches for Emergency Release

In some cases, emergency release can be activated the user (for instance, responsive to a press of the button 770) and continue being engaged without further input from the user. For instance, the emergency release can include a ratchet, clutch, or the like.

Any of the emergency releases described herein can be modified to accommodate various robotic surgery systems. Such systems include, for example, a multi-arm (such as, a three-arm) robotic surgery system. In some cases, the emergency release can be activated by pushing the housing of an instrument manipulator (such as, in a direction away from the surgical site). This approach can be applicable to a robotic surgery system in which instruments are loaded from the side (such as, at an angle to the vertical direction). The emergency release can be activated responsive to the housing of an instrument manipulator being pushed at a predefined force threshold. This may prevent unintended or accidental activation of the emergency release. For example, the emergency release can be configured so that the force feedback created by operating the surgical instrument at the surgical site does not result in activation of the emergency release. The predefined force threshold can be selected so that an unreasonable amount of force to activate the emergency release is not required (thereby allowing the user to activate the emergency release without applying undue force). In some instances, the predefined force threshold can be about 100 N or less.

In some implementations, the emergency release mechanism can be activated by operating an instrument, which can be a custom instrument. For instance, the instrument can be inserted into a channel in the central unit and be configured to engage with the first actuator (such as, include a nut to engage with the leadscrew to detach the instrument manipulator from the leadscrew by turning the instrument). The instrument can be configured so that upon operation of the instrument, the housing of an instrument manipulator moves away from the surgical site. In some cases, the instrument can be operated by pushing and pulling the instrument, turning the instrument, pumping the instrument, activating one or more gears, or the like once the instrument is engaged with the first actuator. In some variations, the instrument can be operated by rotating the instrument once the instrument is engaged with the first actuator. The instrument can be engaged to the first actuator via an access located on an exterior surface of the housing of the instrument manipulator. For example, the access can be located in the area behind the instrument manipulator.

In some implementations, the one or more surgical instruments can be configured to automatically detach from the one or more instrument manipulators upon the one or more instrument manipulators reaching a particular location or position away from the surgical site. Such automatic detachment can occur subsequent to manual retraction caused by emergency release. The point at which the one or more surgical instruments automatically disengage from the surgical manipulator will invariably depend on the characteristics or configuration of the robotic surgery system. For instance, the one or more instruments can be configured to automatically detach responsive to the one or more instrument manipulators reaching the stopper, as described herein. In robotic surgery system in which one or more instruments are loaded from the side, the location of automatic detachment of the one or more instruments can be a particular depth to which the one or more instrument has been retracted. Detaching the one or more surgical instruments from the surgical manipulator can cause the one or more surgical instruments to straighten, as described herein.

### Sterile Barrier

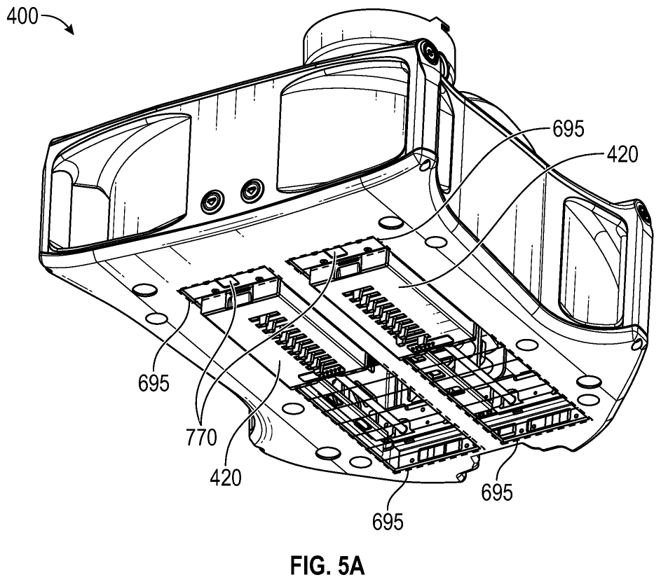

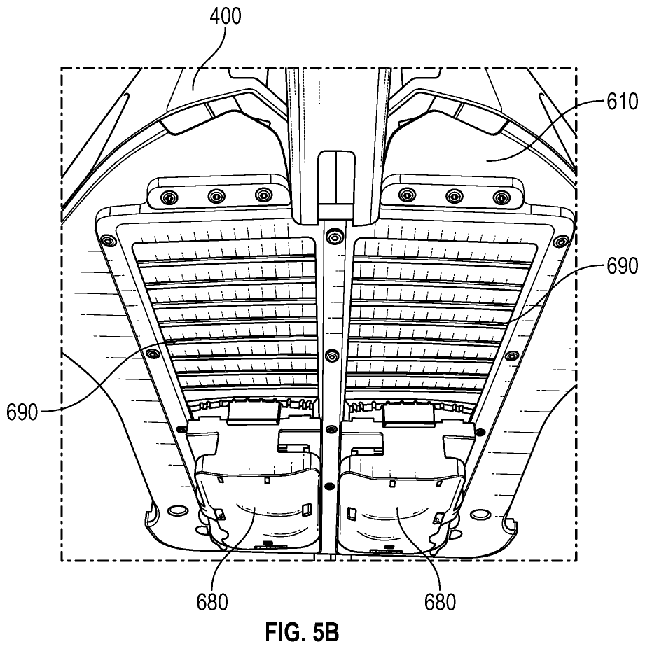

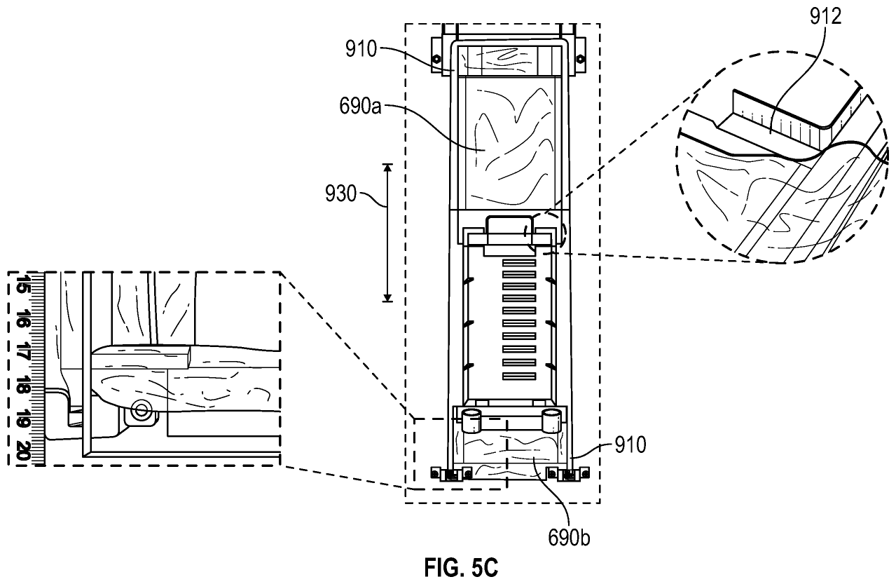

The instrument manipulator 700 can include one or more instrument interfaces 420, as shown in FIG. 5A. One or more sterile adapters 680 can be removably attached to the instrument interfaces 420 (as illustrated in FIG. 5B), and one or more instruments 364 can be supported by the sterile adapter(s) 680 mounted on the instrument interface(s) 420. For example, when a single sterile adapter 680 is used to attach to the instrument interfaces 420, the sterile adapter 680 can be configured to (for example, sized and shaped) to coincide or substantially coincide with the size and shape of the instrument interfaces 420. Sterile adapter 680 can have the same or substantially similar length and width as the instrument interface 420. It may be beneficial to establish and maintain sterility of one or more components of the instrument manipulator 700, such as of the instrument interface(s) 420.

To this end, the central unit 400 can be covered with a sterile barrier that can include a coupler 610 (shown in FIG. 5B) configured to be positioned on the bottom surface of the central unit 400. The coupler 610 can be shaped as a tray and be removably attached to the bottom surface (such as, by using one or more fasteners, which can be magnetic). Additional details of maintaining sterility of the components or the robotic surgery system are disclosed in U.S. Pat. No. 10,881,477, the entire disclose of which is incorporated by reference and should be considered part of this specification.

As shown in FIG. 5B, the coupler 610 can include one or more openings (sometimes referred to as windows) to expose the one or more instrument interfaces 420 of the instrument manipulators 700. The openings (shown as 695 in FIG. 5A) can be moveable to facilitate the linear movement of the one or more instrument manipulators 700 as described herein. To maintain sterility of the one or more surgical instruments (such as, preventing any part of a surgical instrument from coming into contact with a non-sterile bottom surface of the central unit 400), the openings can include flexible portions 690 (sometimes referred to as bellows), which can expand and compress to facilitate movement of the instrument manipulators 700.

The one or more bellows 690 can be configured to create a barrier between the exterior and the interior portions of the central unit 400. For example, the one or more bellows 690 can be positioned to cover the opening(s) (see 695 in FIG. 5A) along the bottom surface of the central unit 400. FIG. 5B shows the one or more bellows 690 covering the openings along the bottom surface of the central unit 400. Because the location of the instrument interfaces 420 along the bottom surface of the central unit can change as the instrument manipulator 700 moves forward toward a surgical site or backward away from the surgical site, the position of the openings along the bottom surface of the central unit 400 can change. Beneficially, the one or more bellows 690 can be configured to expand as the one or more instrument manipulators 700 move forward toward the surgical site and contract as the one or more instrument manipulators 700 move backward away from the surgical site. In some cases, contraction of the one or more bellows 690 is caused by movement toward the surgical side and expansion is caused by the opposite movement. This can allow the one or more bellows 690 to cover the openings along the bottom surface of the central unit 400 notwithstanding the changing position or size of the openings.

In some cases, in place of the one or more bellows 690, flexible material, such as plastic, can be used. With reference to FIG. 5C, the coupler 610 can include a frame or carriage 910 and flexible portions 690A and 690B (shown as being made from transparent material). The flexible portions can be positioned on opposite ends of the carriage 910. Movement of the instrument manipulator causes one flexible portion to contract and the other to expand. The flexible portions can expand or contract as the instrument manipulator 700 moves forward toward a surgical site or backward away from the surgical site. This can allow the one or more flexible portions 690A and 690B to cover the openings along the bottom surface of the central unit 400 notwithstanding the position or size of the openings 695.

The carriage 910 can facilitate expansion or contraction of the flexible portions 690A and 690B as the instrument manipulator 700 moves forward toward a surgical site or backward away from the surgical site. For example, the carriage 910 can include one or more rails configured to guide the flexible portions 690A and 690B as the flexible portions expand and contract. As the instrument manipulator 700 moves forward toward the surgical site or backward away from the surgical site, the one or more rails of the carriage 910 can guide the flexible portions 690A and 690B along the one or more rails in the directions shown by the arrow 930. The flexible portions 690A and 690B can be attached (for instance, glued or welded) to one or more partitions 912 configured to move along the one or more rails. In some cases, each of the flexible portions 690A and 690B is attached to a respective partition 912. Two rails can be positioned on the opposite sides of the carriage 910.

The flexible portions 690A and 690B can be transparent, as shown in FIG. 5C. Beneficially, the transparent properties can allow visualization of the bottom surface of the instrument manipulator 700. For example, the one or more buttons 770 of the emergency release 740 can be visualized through the transparent material. Certain implementations can include non-transparent flexible portions (such as, illustrated in FIG. 5B). The non-transparent portions may block the one or more buttons 770. This may prevent quick and convenient access to the one or more buttons 770. Because of this, nontransparent portions can include a marker configured to indicate the location of the one or more buttons 770 relative to the nontransparent portions. For example, the marker can include a visual marker or a tactile marker indicating the location of the one more buttons 770 along the bottom surface of the instrument manipulator 700. Beneficially, the one or more buttons 770 disclosed herein can be accessed and activated through the nontransparent portions.

### Other Variations

Those skilled in the art will appreciate that, in some embodiments, additional components and/or steps can be utilized, and disclosed components and/or steps can be combined or omitted. For example, although some embodiments are described in connection with a robotic surgery system, the disclosure is not so limited. Systems, devices, and methods described herein can be applicable to medical devices and medical procedures in general, among other uses.

The foregoing description details certain embodiments of the systems, devices, and methods disclosed herein. It will be appreciated, however, that no matter how detailed the foregoing appears in text, the systems, devices, and methods can be practiced in many ways. The use of particular terminology when describing certain features or aspects of the disclosure should not be taken to imply that the terminology is being redefined herein to be restricted to including any specific characteristics of the features or aspects of the technology with which that terminology is associated.

It will be appreciated by those skilled in the art that various modifications and changes can be made without departing from the scope of the described technology. Such modifications and changes are intended to fall within the scope of the embodiments. It will also be appreciated by those of skill in the art that parts included in one embodiment are interchangeable with other embodiments; one or more parts from a depicted embodiment can be included with other depicted embodiments in any combination. For example, any of the various components described herein and/or depicted in the figures can be combined, interchanged, or excluded from other embodiments.

With respect to the use of substantially any plural and/or singular terms herein, those having skill in the art can translate from the plural to the singular and/or from the singular to the plural as is appropriate to the context and/or application. The various singular/plural permutations can be expressly set forth herein for sake of clarity.

Directional terms used herein (for example, top, bottom, side, up, down, inward, outward, etc.) are generally used with reference to the orientation or perspective shown in the figures and are not intended to be limiting. For example, positioning “above” described herein can refer to positioning below or on one of sides. Thus, features described as being “above” may be included below, on one of sides, or the like.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (for example, the bare recitation of “two recitations,” without other modifiers, typically means at least two recitations, or two or more recitations).

The term “comprising” as used herein is synonymous with “including,” “containing,” or “characterized by,” and is inclusive or open-ended and does not exclude additional, unrecited elements or method steps.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Language of degree used herein, such as the terms “approximately,” “about,” “generally,” and “substantially” as used herein represent a value, amount, or characteristic close to the stated value, amount, or characteristic that still performs a desired function and/or achieves a desired result. For example, the terms “approximately”, “about”, “generally,” and “substantially” may refer to an amount that is within less than 10% of, within less than 5% of, within less than 1% of, within less than 0.1% of, and/or within less than 0.01% of the stated amount.

It will be further understood by those within the art that any disjunctive word and/or phrase presenting two or more alternative terms, whether in the description, claims, or drawings, can be understood to contemplate the possibilities of including one of the terms, either of the terms, or both terms. For example, the phrase “A or B” will be understood to include the possibilities of “A” or “B” or “A and B.” Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

The above description discloses embodiments of systems, apparatuses, devices, methods, and materials of the present disclosure. This disclosure is susceptible to modifications in the components, parts, elements, steps, and materials, as well as alterations in the fabrication methods and equipment. Such modifications will become apparent to those skilled in the art from a consideration of this disclosure or practice of the disclosure. Consequently, it is not intended that the disclosure be limited to the specific embodiments disclosed herein, but that it cover all modifications and alternatives coming within the scope and spirit of the subject matter embodied in the following claims.

What is claimed is:

1. An instrument manipulator for a robotic surgery system, the instrument manipulator comprising:
a housing configured to support at least one surgical instrument;
an actuator configured to move the housing forward toward a surgical site and backward away from the surgical site;
a release connected to the housing and configured to:
engage with the actuator in a first configuration to allow the housing and the at least one surgical instrument to move forward toward the surgical site and backward away from the surgical site responsive to activation of the actuator; and
disengage from the actuator in a second configuration to allow a user to manually move the housing and the at least one surgical instrument backward away from the surgical site; and
a user interface positioned at least partially on an exterior surface of the housing and configured to permit the user to transition the release from the first configuration to the second configuration, thereby facilitating retraction of the at least one surgical instrument away from the surgical site.

2. The instrument manipulator of claim 1, wherein manual movement of the housing backward is not permitted in the first configuration as a result of the engagement of the release with the actuator.

3. The instrument manipulator of claim 1, wherein the actuator comprises a leadscrew.

4. The instrument manipulator of claim 3, further comprising a motor configured to rotate the leadscrew to cause the leadscrew to advance and retract the housing.

5. The instrument manipulator of claim 1, wherein the release comprises at least one of a half nut, a dual split nut, a torsion spring, or a radial split nut.

6. The instrument manipulator of claim 1, wherein the release comprises a pin and at least one spring, wherein the at least one spring is configured to extend the pin vertically upward when the release is in the second configuration.

7. The instrument manipulator of claim 6, further comprising a guide configured to engage with the pin to facilitate retraction of the housing away from the surgical site.

8. The instrument manipulator of claim 7, wherein the guide comprises a cutout configured to limit movement of the pin.

9. The instrument manipulator of claim 7, wherein the guide comprises a rail.

10. The instrument manipulator of claim 1, wherein the user interface comprises a button.

11. A central unit comprising at least one instrument manipulator of claim 1.

12. A method of manually retracting an instrument manipulator of a robotic surgery system, the method comprising:
activating an emergency release of the instrument manipulator to cause the emergency release to disengage from an actuator of the instrument manipulator; and
while the emergency release is activated, manually moving the instrument manipulator and at least one surgical instrument supported by the instrument manipulator backward away from a surgical site.

13. The method of claim 12, further comprising removing the at least one surgical instrument from the instrument manipulator subsequent to manually moving the instrument manipulator.

14. The method of claim 13, wherein removing the at least one surgical instrument from the instrument manipulator is performed subsequent to manually retracting the instrument manipulator to a threshold distance away from the surgical site.

15. The method of claim 14, wherein the threshold distance is defined by a stopper of the instrument manipulator configured to limit backward movement of the at least one surgical instrument.

16. The method of claim 12, wherein activating the emergency release of the instrument manipulator comprises pressing a button located on an exterior surface of the instrument manipulator.

17. The method of claim 16, further comprising reengaging the emergency release to the actuator by releasing the button, thereby allowing manipulation of the at least one surgical instrument by the robotic surgery system.

18. The method of claim 12, wherein the actuator comprises a leadscrew.

19. The method of claim 12, wherein the emergency release comprises a pin configured to be biased into a deactivated configuration by a spring, and wherein in the deactivated configuration, manual movement of the instrument manipulator is prevented.

20. The method of claim 12, wherein the emergency release comprises at least one of a half nut, a dual split nut, a torsion spring, or a radial split nut.
